(19) United States
(12) Patent Application Publication
(10) Pub. No.: US 2012/0231013 A1
(43) Pub. Date: Sep. 13, 2012

(54) POLYPEPTIDES THAT BIND TISSUE INHIBITOR OF METALLOPROTEINASE TYPE THREE (TIMP-3), COMPOSITIONS AND METHODS

(76) Inventors: Roy A. Black, Seattle, WA (US); Peng Li, San Francisco, CA (US); Joshua Silverman, Sunnyvale, CA (US); Chadwick T. King, North Vancouver (CA)

(21) Appl. No.: 13/388,242

(22) PCT Filed: Jul. 29, 2010
(86) PCT No.: PCT/US2010/043737

§ 371 Date: May 9, 2012

Related U.S. Application Data
(60) Provisional application No. 61/230,445, filed on Jul. 31, 2009.
(60) Provisional application No. 61/366,783, filed on Jul. 22, 2010.

(51) Int. Cl. A61K 38/57; C12N 5/071; A61K 39/395; A61P 19/02; A61P 9/00; C07K 14/81; C07K 16/40

(52) U.S. Cl. 424/158.1; 530/350; 435/375; 530/389.8; 514/1.1; 514/16.8; 514/16.4

(57) ABSTRACT

The present invention relates to TIMP-3 binding compositions, methods of producing such compositions, and methods of using such compositions, including in the treatment of various conditions.

### CROSS REFERENCE TO RELATED APPLICATIONS

This application claims the benefit under 35 U.S.C. §119 of U.S. Provisional Application Ser. No. 61/230,445, filed Jul. 31, 2009, and U.S. Provisional Application Ser. No. 61/366,783, filed Jul. 22, 2010, which are hereby incorporated by reference in its entirety.

### REFERENCE TO THE SEQUENCE LISTING

The present application is being filed along with a Sequence Listing in electronic format. The Sequence Listing is provided as a file entitled A-1487-WO-PCT_Seq_List.txt, created Jul. 27, 2010, which is 39.9 KB in size. The information in the electronic format of the Sequence Listing is incorporated herein by reference in its entirety.

### FIELD OF THE INVENTION

The present invention relates in general to metalloproteinase inhibitors and to proteins that bind thereto. In particular, the invention relates to tissue inhibitor of metalloproteinase 3 (“TIMP-3”) and its ability to bind a scavenger receptor low density lipoprotein-related protein 1 (“LRP-1”).

### BACKGROUND OF THE INVENTION

Connective tissues and articular cartilage are maintained in dynamic equilibrium by the opposing effects of extracellular matrix synthesis and degradation. Degradation of the matrix is brought about primarily by the enzymatic action of metalloproteinases, including matrix metalloproteinases (MMPs) and disintegrin-metalloproteinases with thrombospondin motifs (ADAMTSs). While these enzymes are important in many natural processes (including development, morphogenesis, bone remodeling, wound healing and angiogenesis), elevated levels are believed to play a role in degradative diseases of connective tissue, including rheumatoid arthritis and osteoarthritis, as well as in cancer and cardiovascular conditions.

Endogenous inhibitors of metalloproteinases include plasma alpha2-macroglobulin and tissue inhibitors of metalloproteinases (TIMPs), of which there are four known to be encoded in the human genome. TIMP-3 inhibits all the major cartilage-degrading metalloproteases, and multiple lines of evidence indicate that it protects cartilage. Addition of the protein to cartilage-explants prevents cytokine-induced degradation, and intra-articular injection reduces cartilage damage in the rat medial meniscal tear model of osteoarthritis. However, development of TIMP-3 as a therapeutic inhibitor of MMP activity has been hampered by challenges in production and short half-life of recombinant forms of TIMP-3.

The LDL receptor-related protein 1 (LRP-1) is a member of the low-density lipoprotein (LDL) receptor gene family with diverse biological roles, including roles in the homeostasis of proteinases and proteinase inhibitors. Similar to other members of the LDL receptor gene family, the structure of LRP-1 is formed of four common structural units in the extracellular domain, each of which unit is further composed of smaller, repeating domains, including cysteine-rich repeats, and epidermal growth factor receptor-like cysteine-rich repeats. More detailed information on the structure of this scavenger receptor is available, for example, in Herz and Strickland, J. Clin. Invest. 108:779 (2001).

LRP-1 has been shown to mediate endocytic clearance of Pro-MMP-2/TIMP-2 complex (Emonard et al., J. Biol. Chem. 279:54944; 2004). Additionally, a chemically-sulfated xylopyranose from beechwood referred to as CaPPS has been shown to increase cartilage levels of TIMP-3 (Troeberg et al., FASEB J 22:3515; 2008). It was suggested that this effect is due to blocking endocytosis of TIMP-3 via LRP-1 because of its similarity to the effects of receptor associated protein (RAP), a general inhibitor of LRP-1, which also increases levels of TIMP-3 in the medium of cultured chondrocytic cells. Accordingly, there is a need in the art to determine whether TIMP-3 interacts directly with LRP-1, to and to define any such interaction with sufficient precision to allow a determination of the effects of any binding between LRP-1 and TIMP-3 on TIMP-3 biological activity. There is a further need to identify agents that can specifically act on such interaction to increase the amount of TIMP-3, in particular, without adversely affecting TIMP-3 biological activity.

### SUMMARY OF THE INVENTION

The invention provides a TIMP-3 binding protein that binds TIMP-3 and inhibits internalization of TIMP-3 by LRP-1. The TIMP-3 binding protein may be an antibody or an LRP-1 peptide. In one aspect, the TIMP-3 binding protein decreases the inhibition of MMP-13 by TIMP-3 by less than 30% (i.e., exhibits relatively little interference with the ability of TIMP-3 to inhibit MMP-13).

The invention further provides a method of increasing TIMP-3 in extracellular matrix by contacting TIMP-3 with a TIMP-3 binding protein that binds TIMP-3 and inhibits internalization of TIMP-3 by LRP-1. The TIMP-3 binding protein may be an antibody or an LRP-1 peptide. In one aspect, the TIMP-3 binding protein of claim decreases the inhibition of MMP-13 by TIMP-3 by less than 30% (i.e., exhibits relatively little interference with the ability of TIMP-3 to inhibit MMP-13). The TIMP-3 is contacted with the TIMP-3 binding protein in vivo, ex vivo or in vitro; contacted with the TIMP-3 binding protein in vivo may be achieved by administering the TIMP-3 binding protein to a mammal.

The invention also provides a method of treating a mammal afflicted with a condition in which matrix metalloproteinases play a deleterious role, comprising administering a TIMP-3 binding protein that inhibits internalization of TIMP-3 by LRP-1 to the mammal. The TIMP-3 binding protein may be an antibody or an LRP-1 peptide. In one aspect, the TIMP-3 binding protein decreases the inhibition of MMP-13 by TIMP-3 by less than 30% (i.e., exhibits relatively little interference with the ability of TIMP-3 to inhibit MMP-13). The condition may be selected from the group consisting of inflammation, cancer, and a condition characterized by excessive degradation of the extracellular matrix; for example, the condition may be selected from the group consisting of osteoarthritis and congestive heart failure.

### DETAILED DESCRIPTION OF THE INVENTION

The present invention provides compositions, kits, and methods relating to polypeptides that bind to TIMP-3, such as naturally-occurring polypeptides (i.e., LRP-1 polypeptides) and fragments thereof, anti-TIMP-3 antibodies, antibody fragments, and antibody derivatives. Also provided are nucleic acids, and derivatives and fragments thereof, comprising a sequence of nucleotides that encodes all or a portion of a polypeptide that binds to TIMP-3, e.g., a nucleic acid encoding all or part of such TIMP-3-binding proteins, plasmids and vectors comprising such nucleic acids, and cells or cell lines comprising such nucleic acids and/or vectors and plasmids. The provided methods include, for example, methods of making, identifying, or isolating molecules that bind to TIMP-3, such as anti-TIMP-3 antibodies, methods of determining whether a molecule binds to TIMP-3, methods of determining whether a molecule agonizes or antagonizes TIMP-3 activity, as well as methods of determining whether a molecule facilitates accumulation of TIMP-3 (for example, in cultures of chondrocyte-like cells or cell lines or in ex vivo cartilage explants).

TIMP-3 is expressed by various cells or tissues in a mammal and is present in the extracellular matrix; the TIMP-3 that is so expressed is referred to herein as “endogenous” TIMP-3. Numerous conditions exist in which it would be advantageous to increase, elevate or enhance the amount of endogenous TIMP-3 in a mammal. Accordingly, also provided herein are methods of making compositions, such as pharmaceutical compositions, comprising a molecule that binds to TIMP-3, and methods for administering a composition comprising a molecule that binds TIMP-3 to a subject, for example, methods for treating a condition by facilitating accumulation of TIMP-3, by increasing the endogenous amount of TIMP-3, by inhibiting the binding of TIMP-3 to cells, by decreasing internalization of TIMP-3, and/or by agonizing a biological activity of TIMP-3, in vivo, ex vivo or in vitro.

Polynucleotide and polypeptide sequences are indicated using standard one- or three-letter abbreviations. Unless otherwise indicated, each polypeptide sequence has an amino terminus at the left and a carboxy terminus at the right; each single-stranded nucleic acid sequence, and the top strand of each double-stranded nucleic acid sequence, has a 5′ terminus at the left and a 3′ terminus at the right. A particular polypeptide or polynucleotide sequence also can be described by explaining how it differs from a reference sequence.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature without human intervention. Thus, a molecule that is chemically synthesized, or synthesized in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A “TIMP-3 agonist” as used herein is a molecule that detectably increases at least one function of TIMP-3, for example, by increasing the amount of TIMP-3 (accumulation) in vitro, ex vivo or in vivo, without significantly decreasing the ability of TIMP-3 to inhibit one or more metalloproteinases. Any assay of a function of TIMP-3 can be used, examples of which are provided herein. Examples of functions of TIMP-3 that can be increased by a TIMP-3 agonist include inhibition of matrix metalloproteinases, including MMP-13. For example, a TIMP-3 agonist may increase the MMP-13-inhibiting activity from a cartilage explant, or a chondrocyte cell culture, or it may enhance the level of TIMP-3 in vivo, thereby increasing the MMP-13 inhibiting activity in vivo. Examples of types of TIMP-3 agonists include, but are not limited to, TIMP-3 binding polypeptides such as polypeptides derived from LRP-1 as well as antigen binding proteins (e.g., TIMP-3 antigen binding proteins), antibodies, antibody fragments, and antibody derivatives.

The terms “peptide,” “polypeptide” and “protein” each refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric.

The term “polypeptide fragment” as used herein refers to a polypeptide that has an amino-terminal and/or carboxy-terminal deletion as compared to a corresponding full-length protein. Fragments can be, for example, at least 5, 6, 7, 8, 9, 10, 11, 12, 13, 14, 15, 20, 50, 70, 80, 90, 100, 150 or 200 amino acids in length. Fragments can also be, for example, at most 1,000, 750, 500, 250, 200, 175, 150, 125, 100, 90, 80, 70, 60, 50, 40, 30, 20, 15, 14, 13, 12, 11, or 10 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence or a tag protein).

Polypeptides of the invention include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties. Analogs include muteins of a polypeptide. For example, single or multiple amino acid substitutions (e.g., conservative amino acid substitutions) may be made in the naturally occurring sequence (e.g., in the portion of the polypeptide outside the domain(s) forming intermolecular contacts). Consensus sequences can be used to select amino acid residues for substitution; those of skill in the art recognize that additional amino acid residues may also be substituted.

A “conservative amino acid substitution” is one that does not substantially change the structural characteristics of the parent sequence (e.g., a replacement amino acid should not tend to break a helix that occurs in the parent sequence, or disrupt other types of secondary structure that characterize the parent sequence or are necessary for its functionality). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The present invention also provides non-peptide analogs of TIMP-3 binding polypeptides. Non-peptide analogs are commonly used in the pharmaceutical industry as drugs with properties analogous to those of the template peptide. These types of non-peptide compound are termed “peptide mimetics” or “peptidomimetics,” see, for example, Fauchere, J. Adv. Drug Res. 15:29 (1986); Veber and Freidinger TINS p. 392 (1985); and Evans et al. J. Med. Chem. 30:1229 (1987), which are incorporated herein by reference. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference), for example, by adding internal cysteine residues capable of forming intramolecular disulfide bridges which cyclize the peptide.

A “variant” of a polypeptide (e.g., an antibody) comprises an amino acid sequence wherein one or more amino acid residues are inserted into, deleted from and/or substituted into the amino acid sequence relative to another polypeptide sequence. Variants of the invention include fusion proteins.

A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and/or glycosylation. Unless otherwise indicated, the term “antibody” includes, in addition to antibodies comprising two full-length heavy chains and two full-length light chains, derivatives, variants, fragments, and muteins thereof, examples of which are described below.

An “antigen binding protein” is a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

The variable regions of naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001).

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen.

An “antibody” refers to an intact immunoglobulin or to an antigen binding portion thereof that competes with the intact antibody for specific binding, unless otherwise specified. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, variable region fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, triabodies and tetrabodies are antibodies comprising three and four polypeptide chains, respectively, and forming three and four antigen binding sites, respectively, which can be the same or different.

Complementarity determining regions (CDRs) and framework regions (FR) of a given antibody may be identified using the system described by Kabat et al. supra; Lefranc et al., supra and/or Honegger and Pluckthun, supra. One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

An antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-TIMP-3 antibody. In another embodiment, all of the CDRs are derived from a human anti-TIMP-3 antibody. In another embodiment, the CDRs from more than one human anti-TIMP-3 antibodies are mixed and matched in a chimeric antibody. For instance, a chimeric antibody may comprise a CDR1 from the light chain of a first human anti-TIMP-3 antibody, a CDR2 and a CDR3 from the light chain of a second human anti-TIMP-3 antibody, and the CDRs from the heavy chain from a third anti-TIMP-3 antibody. Other combinations are possible and are included within the embodiments of the invention.

Further, the framework regions may be derived from one of the same anti-TIMP-3 antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind TIMP-3). See, e.g., U.S. Pat. No. 4,816,567 and Morrison, 1985, Science 229:1202-07.

An “LRP-1 inhibitory antibody” is an antibody that inhibits the interaction of TIMP-3 with LRP-1 when an excess of the anti-TIMP-3 antibody reduces the amount of interaction by at least about 20% using an assay such as those described herein in the Examples. In various embodiments, the antigen binding protein reduces the interaction of TIMP-3 with LRP-1 TIMP-3 by at least 30%, 40%, 50%, 60%, 70%, 75%, 80%, 85%, 90%, 95%, 97%, 99%, and 99.9%. In other embodiments, the antigen binding protein increases the accumulation of TIMP-3 by at least 10%, 20%, 30%, 40%, 50%, 60%, 70%, 75%, 80%, 85%, 90%, 95%, 97%, 99%, and 99.9%.

Some LRP-1 inhibitory antibodies may inhibit binding of TIMP-3 to LRP-1, and also interfere with the ability of TIMP-3 to inhibit MMPs. However, some LRP-1 inhibitory antibodies inhibit binding of TIMP-3 to LRP-1, without adversely affecting the ability of TIMP-3 to inhibit MMPs; such antibodies are referred to herein as “agonizing” or “agonistic” antibodies. Agonistic antibodies will generally result in a less than 40%, 30%, 20%, 10%, 5%, 1% or less than 1% decrease in MMP-inhibitory activity.

Fragments or analogs of antibodies can be readily prepared by those of ordinary skill in the art following the teachings of this specification and using techniques well-known in the art. Amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

A “CDR grafted antibody” is an antibody comprising one or more CDRs derived from an antibody of a particular species or isotype and the framework of another antibody of the same or different species or isotype.

A “multi-specific antibody” is an antibody that recognizes more than one epitope on one or more antigens. A subclass of this type of antibody is a “bi-specific antibody” which recognizes two distinct epitopes on the same or different antigens.

An antigen binding protein “specifically binds” to an antigen (e.g., human TIMP-3) if it binds to the antigen with a dissociation constant of 1 nanomolar or less.

An “antigen binding domain,” “antigen binding region,” or “antigen binding site” is a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

An “epitope” is the portion of a molecule that is bound by an antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein). Epitope may also be used when referring to the portion of a molecule that is bound by a binding protein other than an antigen binding protein, and may similarly comprise linear, contiguous, or non-contiguous portions of the molecule.

Analysis of protein sequences and three-dimensional structures have revealed that many proteins are composed of a number of discrete units referred to as “monomer domains.” The majority of discrete monomer domain proteins is extracellular or constitutes the extracellular parts of membrane-bound proteins.

An important characteristic of a discrete monomer domain is its ability to fold independently or with some limited assistance. Limited assistance can include assistance of a chaperonin(s) (e.g., a receptor-associated protein (RAP)). The presence of a metal ion(s) also offers limited assistance. The ability to fold independently prevents misfolding of the domain when it is inserted into a new protein environment. This characteristic has allowed discrete monomer domains to be evolutionarily mobile. As a result, discrete domains have spread during evolution and now occur in otherwise unrelated proteins. Some domains, including the fibronectin type III domains and the immunoglobin-like domain, occur in numerous proteins, while other domains are only found in a limited number of proteins.

Proteins that contain these domains are involved in a variety of processes, such as cellular transporters, cholesterol movement, signal transduction and signaling functions which are involved in development and neurotransmission. See Herz, Trends in Neurosciences 24:193 (2001); Goldstein and Brown, Science 292:1310 (2001). The function of a discrete monomer domain is often specific but it also contributes to the overall activity of the protein or polypeptide. For example, the LDL-receptor class A domain (also referred to as a class A module, a complement type repeat or an A-domain) is involved in ligand binding while the gamma-carboxyglumatic acid (Gla) domain which is found in the vitamin-K-dependent blood coagulation proteins is involved in high-affinity binding to phospholipid membranes. Other discrete monomer domains include, e.g., the epidermal growth factor (EGF)-like domain in tissue-type plasminogen activator which mediates binding to liver cells and thereby regulates the clearance of this fibrinolytic enzyme from the circulation and the cytoplasmic tail of the LDL-receptor which is involved in receptor-mediated endocytosis.

Individual proteins can possess one or more discrete monomer domains. These proteins are often called mosaic proteins. For example, members of the LDL-receptor family contain four major structural domains: the cysteine rich A-domain repeats, epidermal growth factor precursor-like repeats, a transmembrane domain and a cytoplasmic domain.

The LDL-receptor family includes members that: 1) are cell-surface receptors; 2) recognize extracellular ligands; and 3) internalize them for degradation by lysosomes. See Hussain et al., Annu. Rev. Nutr. 19:141 (1999). For example, some members include very-low-density lipoprotein receptors (VLDL-R), apolipoprotein E receptor 2, LDLR-related protein (LRP or LRP-1) and megalin. Family members have the following characteristics: 1) cell-surface expression; 2) extracellular ligand binding consisting of A-domain repeats; 3) requirement of calcium for ligand binding; 4) recognition of receptor-associated protein and apolipoprotein (apo) E; 5) epidermal growth factor (EGF) precursor homology domain containing YWTD repeats; 6) single membrane-spanning region; and 7) receptor-mediated endocytosis of various ligands. See Hussain, supra. However, the members bind several structurally dissimilar ligands.

“LRP-1 polypeptides” or “LRP-1 peptides” as used herein are polypeptides (or peptides that are related to LRP-1 by being fragments of LRP-1, for example, fragments of the extracellular domain (or “ectodomain”) of LRP-1. The polypeptides may comprise one (or more) ligand-binding clusters of LRP-1 (see, for example, Herz and Strickland, supra, and the Examples herein). The polypeptides (or peptides) may comprise a portion of a ligand binding cluster, for example, a discrete monomer domain such as an A-domain. The polypeptides may further consist of multimers (for example, dimers or trimers, or higher-order multimers) of discrete monomer domains such as an A-domain. The multimers may include more than one structurally distinct (i.e., having differing amino acid sequences) monomer domain, or may include multiple repeats of a single monomer domain, or may include both multiple repeating monomer domains and structurally distinct domains.

An “LRP-1 inhibitory polypeptide” is a polypeptide that inhibits the interaction of TIMP-3 with LRP-1 when an excess of the polypeptide reduces the amount of interaction by at least about 20% using an assay such as those described herein in the Examples. In various embodiments, the polypeptide reduces the interaction of TIMP-3 with LRP-1 by at least 30%, 40%, 50%, 60%, 70%, 75%, 80%, 85%, 90%, 95%, 97%, 99%, and 99.9%. In other embodiments, the polypeptide increases accumulation of TIMP-3 by at least 10%, 20%, 30%, 40%, 50%, 60%, 70%, 75%, 80%, 85%, 90%, 95%, 97%, 99%, and 99.9%.

Some LRP-1 inhibitory polypeptides may inhibit binding of TIMP-3 to LRP-1, and also interfere with the ability of TIMP-3 to inhibit MMPs. However, some LRP-1 inhibitory polypeptides inhibit binding of TIMP-3 to LRP-1, without adversely affecting the ability of TIMP-3 to inhibit MMPs; such polypeptides are referred to herein as “agonizing” or “agonistic” polypeptides. Agonistic polypeptides will generally result in a less than 40%, 30%, 20%, 10%, 5%, 1% or less than 1% decrease in MMP-inhibitory activity.

The “percent identity” of two polynucleotide or two polypeptide sequences is determined by comparing the sequences using the GAP computer program (a part of the GCG Wisconsin Package, version 10.3 (Accelrys, San Diego, Calif.)) using its default parameters.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody, or a fragment, derivative, mutein, or variant thereof, of the invention.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

### Antigen Binding Proteins

In one aspect, the present invention provides antigen binding proteins (e.g., antibodies, antibody fragments, antibody derivatives, antibody muteins, and antibody variants) that bind to TIMP-3, e.g., human TIMP-3.

Different antigen binding proteins may bind to different domains or epitopes of TIMP-3 or act by different mechanisms of action. Examples include but are not limited to antigen binding proteins that interfere with the ability of TIMP-3 to bind LRP-1 or that inhibit the ability of TIMP-3 to inhibit MMPs. Further examples include antigen binding proteins that interfere with the ability of TIMP-3 to bind LRP-1 but do not inhibit the ability of TIMP-3 to inhibit MMPs (i.e., TIMP-3 agonists). Discussions herein of particular mechanisms of action for TIMP-3-binding antigen binding proteins in treating particular diseases are illustrative only, and the methods presented herein are not bound thereby.

Other derivatives of anti-TIMP-3 antibodies within the scope of this invention include covalent or aggregative conjugates of anti-TIMP-3 antibodies, or fragments thereof, with other proteins or polypeptides, such as by expression of recombinant fusion proteins comprising heterologous polypeptides fused to the N-terminus or C-terminus of an anti-TIMP-3 antibody polypeptide. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of antigen binding protein (e.g., poly-His). An antigen binding protein also can be linked to the FLAG® peptide Asp-Tyr-Lys-Asp-Asp-Asp-Asp-Lys (DYKDDDDK) (SEQ ID NO:2) as described in Hopp et al., Bio/Technology 6:1204, 1988, and U.S. Pat. No. 5,011,912. The FLAG® peptide is highly antigenic and provides an epitope reversibly bound by a specific monoclonal antibody (mAb), enabling rapid assay and facile purification of expressed recombinant protein. Reagents useful for preparing fusion proteins in which the FLAG® peptide is fused to a given polypeptide are commercially available (Sigma-Aldrich, St. Louis Mo.).

Oligomers that contain one or more antigen binding proteins may be employed as TIMP-3 agonists. Oligomers may be in the form of covalently-linked or non-covalently-linked dimers, trimers, or higher oligomers. Oligomers comprising two or more antigen binding proteins are contemplated for use, with one example being a homodimer. Other oligomers include heterodimers, homotrimers, heterotrimers, homotetramers, heterotetramers, etc.

One embodiment is directed to oligomers comprising multiple antigen binding proteins joined via covalent or non-covalent interactions between peptide moieties fused to the antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular embodiments, the oligomers comprise from two to four antigen binding proteins. The antigen binding proteins of the oligomer may be in any form, such as any of the forms described above, e.g., variants or fragments. Preferably, the oligomers comprise antigen binding proteins that have TIMP-3 binding activity.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, PNAS USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

One embodiment of the present invention is directed to a dimer comprising two fusion proteins created by fusing a TIMP-3 binding fragment of an anti-TIMP-3 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of an anti-TIMP-3 antibody may be substituted for the variable portion of an antibody heavy and/or light chain.

Alternatively, the oligomer is a fusion protein comprising multiple antigen binding proteins, with or without peptide linkers (spacer peptides). Among the suitable peptide linkers are those described in U.S. Pat. Nos. 4,751,180 and 4,935,233.

Another method for preparing oligomeric antigen binding proteins involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising an anti-TIMP-3 antibody fragment or derivative fused to a leucine zipper peptide are expressed in suitable host cells, and the soluble oligomeric anti-TIMP-3 antibody fragments or derivatives that form are recovered from the culture supernatant.

In one aspect, the present invention provides antigen binding proteins that interfere with the binding of TIMP-3 to LRP-1. Such antigen binding proteins can be made against TIMP-3, or a fragment, variant or derivative thereof, and screened in conventional assays for the ability to interfere with the binding of TIMP-3 to LRP-1. Examples of suitable assays are disclosed herein, and include assays that test the antigen binding proteins for the ability to inhibit binding to LRP-1, or that test antigen binding proteins for the ability to increase the amount of TIMP-3, for example in culture or ex vivo. Additional assays that test the antigen binding proteins include those that qualitatively or quantitatively compare the binding of an antigen binding protein to a TIMP-3 polypeptide to the binding of a known antigen binding protein to a TIMP-3 polypeptide, several examples of which are disclosed herein.

In another aspect, the present invention provides an antigen binding protein that demonstrates species selectivity. In one embodiment, the antigen binding protein binds to one or more mammalian TIMP-3, for example, to human TIMP-3 and one or more of mouse, rat, guinea pig, hamster, gerbil, cat, rabbit, dog, goat, sheep, cow, horse, camel, and non-human primate TIMP-3. In another embodiment, the antigen binding protein binds to one or more primate TIMP-3, for example, to human TIMP-3 and one or more of cynomologous, marmoset, rhesus, tamarin and chimpanzee TIMP-3. In another embodiment, the antigen binding protein binds specifically to human, cynomologous, marmoset, rhesus, tamarin or chimpanzee TIMP-3. In another embodiment, the antigen binding protein does not bind to one or more of mouse, rat, guinea pig, hamster, gerbil, cat, rabbit, dog, goat, sheep, cow, horse, camel, and non-human primate TIMP-3. In another embodiment, the antigen binding protein does not bind to a New World monkey species such as a marmoset.

In another embodiment, the antigen binding protein does not exhibit specific binding to any naturally occurring protein other than TIMP-3. In another embodiment, the antigen binding protein does not exhibit specific binding to any naturally occurring protein other than mammalian TIMP-3. In another embodiment, the antigen binding protein does not exhibit specific binding to any naturally occurring protein other than primate TIMP-3. In another embodiment, the antigen binding protein does not exhibit specific binding to any naturally occurring protein other than human TIMP-3. In another embodiment, the antigen binding protein specifically binds to TIMP-3 from at least one non-human primate, for example, cynomologous monkey, and human TIMP-3. In another embodiment, the antigen binding protein specifically binds to non-human primate, cynomologous monkey, and human TIMP-3 with a similar binding affinity. In another embodiment, the antigen binding protein blocks an activity of non-human primate, cynomologous monkey, and human TIMP-3. In another embodiment, the antigen binding protein has a similar IC₅₀ or EC₅₀ against non-human primate, cynomologous monkey, and human TIMP-3 in an assay as described herein.

One may determine the selectivity of an antigen binding protein for a TIMP-3 using methods well known in the art and following the teachings of the specification. For example, one may determine the selectivity using Western blot, FACS, ELISA or RIA.

Antigen-binding fragments of antigen binding proteins of the invention may be produced by conventional techniques. Examples of such fragments include, but are not limited to, Fab and F(ab′)₂ fragments. Antibody fragments and derivatives produced by genetic engineering techniques also are contemplated.

Additional embodiments include chimeric antibodies, e.g., humanized versions of non-human (e.g., murine) monoclonal antibodies. Such humanized antibodies may be prepared by known techniques, and offer the advantage of reduced immunogenicity when the antibodies are administered to humans. In one embodiment, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of chimeric and further engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody.

Techniques for humanizing antibodies are discussed in, e.g., U.S. patent application Ser. No. 10/194,975 (published Feb. 27, 2003), U.S. Pat. Nos. 5,869,619, 5,225,539, 5,821,337, 5,859,205, Padlan et al., 1995, FASEB J. 9:133-39, and Tamura et al., 2000, J. Immunol. 164:1432-41.

Procedures have been developed for generating human or partially human antibodies in non-human animals. For example, mice in which one or more endogenous immunoglobulin genes have been inactivated by various means have been prepared. Human immunoglobulin genes have been introduced into the mice to replace the inactivated mouse genes. Antibodies produced in the animal incorporate human immunoglobulin polypeptide chains encoded by the human genetic material introduced into the animal. In one embodiment, a non-human animal, such as a transgenic mouse, is immunized with a TIMP-3 polypeptide, such that antibodies directed against the TIMP-3 polypeptide are generated in the animal. One example of a suitable immunogen is a soluble human TIMP-3, such as a polypeptide comprising an LRP-1 binding domain of TIMP-3, or other immunogenic fragment TIMP-3. Another example of a suitable immunogen is cells expressing high levels of TIMP-3, or cell membrane preparations therefrom.

Examples of techniques for production and use of transgenic animals for the production of human or partially human antibodies are described in U.S. Pat. Nos. 5,814,318, 5,569,825, and 5,545,806, Davis et al., 2003, Production of human antibodies from transgenic mice in Lo, ed. Antibody Engineering: Methods and Protocols, Humana Press, NJ:191-200, Kellermann et al., 2002, Curr Opin Biotechnol. 13:593-97, Russel et al., 2000, Infect Immun. 68:1820-26, Gallo et al., 2000, Eur J Immun. 30:534-40, Davis et al., 1999, Cancer Metastasis Rev. 18:421-25, Green, 1999, J Immunol Methods. 231:11-23, Jakobovits, 1998, Adv Drug Deliv Rev 31:33-42, Green et al., 1998, J Exp Med. 188:483-95, Jakobovits A, 1998, Exp. Opin. Invest. Drugs. 7:607-14, Tsuda et al., 1997, Genomics 42:413-21, Mendez et al., 1997, Nat Genet. 15:146-56, Jakobovits, 1994, Curr Biol. 4:761-63, Arbones et al., 1994, Immunity. 1:247-60, Green et al., 1994, Nat Genet. 7:13-21, Jakobovits et al., 1993, Nature 362:255-58, Jakobovits et al., 1993, Proc Natl Acad Sci USA. 90:2551-55. Chen, J. et al., 1993, Int Immunol 5: 647-656, Choi et al., 1993, Nature Genetics 4: 117-23, Fishwild et al., 1996, Nat Biotechnol 14: 845-51, Harding et al., 1995, Ann NY Acad Sci, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Int Rev Immunol 13: 65-93, Neuberger, 1996, Nat Biotechnol 14: 826, Taylor et al., 1992, Nucleic Acids Research 20: 6287-95, Taylor et al., 1994, Int Immunol 6: 579-91, Tomizuka et al., 1997, Nat Gen 16: 133-43, Tomizuka et al., 2000, Proc Natl Acad Sci USA. 97: 722-27, Tuaillon et al., 1993, Proc Natl Acad Sci USA. 90: 3720-24, and Tuaillon et al., 1994, J Immunol 152: 2912-20. These and other examples are also discussed in U.S. Patent application publication 2007-0098715, published May 3, 2007.

In another aspect, the present invention provides monoclonal antibodies that bind to TIMP-3. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In one embodiment, a hybridoma cell line is produced by immunizing an animal (e.g., a transgenic animal having human immunoglobulin sequences) with a TIMP-3 immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds a TIMP-3 polypeptide. Such hybridoma cell lines, and anti-TIMP-3 monoclonal antibodies produced by them, are encompassed by the present invention.

Monoclonal antibodies secreted by a hybridoma cell line can be purified using any technique known in the art. Hybridomas or mAbs may be further screened to identify mAbs with particular properties, such as the ability to block a TIMP-3 induced activity. Examples of such screens are provided in the examples below.

Monoclonal antibodies can also be produced using a process referred to as genetic immunization. For example, a nucleic acid encoding the antigen of interest can be incorporated into a viral vector (such as an adenoviral vector). The resulting vector is then used to develop an immune response against the antigen of interest in a suitable host animal (for example, a non-obese diabetic, or NOD, mouse). This techniques is substantially described by Ritter et al., Biodrugsl6(1): 3-10 (2002), the disclosure of which is incorporated by reference herein.

In one aspect, the present invention provides antigen-binding fragments of an anti-TIMP-3 antibody of the invention. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., 2002, Biochem. Soc. Trans. 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

Antigen binding proteins (e.g., antibodies, antibody fragments, and antibody derivatives) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16. Moreover, if an IgG4 is desired, it may also be desired to introduce a point mutation (CPSCP->CPPCP) in the hinge region as described in Bloom et al., 1997, Protein Science 6:407, incorporated by reference herein) to alleviate a tendency to form intra-H chain disulfide bonds that can lead to heterogeneity in the IgG4 antibodies.

Moreover, techniques for deriving antigen binding proteins having different properties (i.e., varying affinities for the antigen to which they bind) are also known. One such technique, referred to as chain shuffling, involves displaying immunoglobulin variable domain gene repertoires on the surface of filamentous bacteriophage, often referred to as phage display. Chain shuffling has been used to prepare high affinity antibodies to the hapten 2-phenyloxazol-5-one, as described by Marks et al., 1992, BioTechnology, 10:779.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J. Mol. Biol. 263:551. Accordingly, such techniques are useful in preparing antibodies to TIMP-3.

In one embodiment, the present invention provides an antigen binding protein that has a low dissociation constant from TIMP-3. In one embodiment, the antigen binding protein has a K_{d} of 100 pM or lower. In another embodiment, the K_{d} is 10 pM or lower; in another embodiment, it is 5 pM or lower, or it is 1 pM or lower. In another embodiment, the K_{d} is substantially the same as an antibody described herein in the Examples. In another embodiment, the antigen binding protein binds to TIMP-3 with substantially the same K_{d} as an antibody described herein in the Examples.

The present invention further provides multi-specific antigen binding proteins, for example, bispecific antigen binding protein, e.g., antigen binding protein that bind to two different epitopes of TIMP-3, or to an epitope of TIMP-3 and an epitope of another molecule, via two different antigen binding sites or regions. Moreover, bispecific antigen binding protein as disclosed herein can comprise a TIMP-3 binding site from one of the herein-described antibodies and a second TIMP-3 binding region from another of the herein-described antibodies, including those described herein by reference to other publications. Alternatively, a bispecific antigen binding protein may comprise an antigen binding site from one of the herein described antibodies and a second antigen binding site from another TIMP-3 antibody that is known in the art, or from an antibody that is prepared by known methods or the methods described herein.

Numerous methods of preparing bispecific antibodies are known in the art, and discussed in U.S. patent application Ser. No. 09/839,632, filed Apr. 20, 2001 (incorporated by reference herein). Such methods include the use of hybrid-hybridomas as described by Milstein et al., 1983, Nature 305:537, and others (U.S. Pat. No. 4,474,893, U.S. Pat. No. 6,106,833), and chemical coupling of antibody fragments (Brennan et al., 1985, Science 229:81; Glennie et al., 1987, J. Immunol. 139:2367; U.S. Pat. No. 6,010,902). Moreover, bispecific antibodies can be produced via recombinant means, for example by using leucine zipper moieties (i.e., from the Fos and Jun proteins, which preferentially form heterodimers; Kostelny et al., 1992, J. Immunol. 148:1547) or other lock and key interactive domain structures as described in U.S. Pat. No. 5,582,996. Additional useful techniques include those described in Kortt et al., 1997, supra; U.S. Pat. No. 5,959,083; and U.S. Pat. No. 5,807,706.

### Uses for TIMP-3 Binding Proteins

TIMP-3 binding proteins can be used, for example, in assays to detect the presence of TIMP-3 or cells expressing TIMP-3, either in vitro or in vivo. The TIMP-3 binding proteins also may be employed in purifying TIMP-3 proteins by immunoaffinity chromatography. Those TIMP-3 binding proteins that additionally can block the interaction of LRP-1 and TIMP-3 may be used to increase the accumulation of TIMP-3, and/or to enhance the endogenous levels of TIMP-3, in vitro, ex vivo or in vivo. TIMP-3 binding proteins that increase the accumulation of TIMP-3 without adversely affecting the ability of TIMP-3 to inhibit MMPs may be used to increase a biological activity of TIMP-3 (i.e., as TIMP-3 agonists). TIMP-3 binding proteins that function as TIMP-3 agonists may be employed in treating any condition in which a greater level of TIMP-3 activity is desired (i.e., conditions in which MMPs and/or other proteinases that are inhibited by TIMP-3 play a role), including but not limited to inflammatory conditions. In one embodiment, a human anti-TIMP-3 monoclonal antibody generated by procedures involving immunization of transgenic mice is employed in treating such conditions.

TIMP-3 binding proteins may be employed in an in vitro procedure, or administered in vivo to increase accumulation of TIMP-3, to elevate endogenous levels of TIMP-3 and/or enhance a TIMP-3-induced biological activity. Disorders caused or exacerbated (directly or indirectly) by TIMP-3-inhibitable proteinases, examples of which are provided herein, thus may be treated. In one embodiment, the present invention provides a therapeutic method comprising in vivo administration of an agonistic TIMP-3 binding protein to a mammal in need thereof in an amount effective for increasing a TIMP-3-induced biological activity. In another embodiment, the present invention provides a therapeutic method comprising in vivo administration of an agonistic TIMP-3 binding protein to a mammal in need thereof in an amount effective for elevating endogenous levels of TIMP-3.

TIMP-3 binding proteins of the invention include partially human and fully human monoclonal antibodies as well as LRP-1 polypeptides or peptides. One embodiment is directed to a human monoclonal antibody that at least partially agonizes an activity TIMP-3. In one embodiment, the antibodies are generated by immunizing a transgenic mouse with a TIMP-3 immunogen. In another embodiment, the immunogen is a human TIMP-3 polypeptide (e.g., a cell transformed or transfected to express TIMP-3, or a cell that naturally expresses TIMP-3). Hybridoma cell lines derived from such immunized mice, wherein the hybridoma secretes a monoclonal antibody that binds TIMP-3, also are provided herein.

Although human, partially human, or humanized antibodies will be suitable for many applications, particularly those involving administration of the antibody to a human subject, other types of antibodies will be suitable for certain applications. The non-human antibodies of the invention can be, for example, derived from any antibody-producing animal, such as mouse, rat, rabbit, goat, donkey, or non-human primate (such as monkey (e.g., cynomologous or rhesus monkey) or ape (e.g., chimpanzee)).

Non-human antibodies of the invention can be used, for example, in in vitro and cell-culture based applications, or any other application where an immune response to the antibody of the invention does not occur, is insignificant, can be prevented, is not a concern, or is desired. In one embodiment, a non-human antibody of the invention is administered to a non-human subject. In another embodiment, the non-human antibody does not elicit an immune response in the non-human subject. In another embodiment, the non-human antibody is from the same species as the non-human subject, e.g., a mouse antibody of the invention is administered to a mouse.

An antibody from a particular species can be made by, for example, immunizing an animal of that species with the desired immunogen (e.g., cells expressing TIMP-3, or a soluble TIMP-3 polypeptide) or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

TIMP-3 binding proteins may be prepared by any of a number of conventional techniques. For example, they may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Any expression system known in the art can be used to make the recombinant polypeptides of the invention. In general, host cells are transformed with a recombinant expression vector that comprises DNA encoding a desired polypeptide. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., 1991, EMBO J. 10: 2821. Appropriate cloning and expression vectors for use with bacterial, fungal, yeast, and mammalian cellular hosts are described by Pouwels et al. (Cloning Vectors: A Laboratory Manual, Elsevier, New York, 1985).

The transformed cells can be cultured under conditions that promote expression of the polypeptide, and the polypeptide recovered by conventional protein purification procedures. One such purification procedure includes the use of affinity chromatography, e.g., over a matrix having all or a portion of TIMP-3 bound thereto. Polypeptides contemplated for use herein include substantially homogeneous recombinant mammalian TIMP-3 binding polypeptides substantially free of contaminating endogenous materials.

TIMP-3 binding proteins may be prepared, and screened for desired properties, by any of a number of known techniques. Certain of the techniques involve isolating a nucleic acid encoding a polypeptide chain (or portion thereof) of a TIMP-3 binding protein of interest (e.g., an anti-TIMP-3 antibody), and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example.

In one aspect, the present invention provides a TIMP-3 binding protein that agonizes an activity of TIMP-3, for example by increasing the accumulation of TIMP-3 or elevating the levels of endogenous TIMP-3. In another embodiment, the antigen binding protein agonizes an activity of TIMP-3 by substantially the same amount as an antibody described herein in the Examples.

In one embodiment, TIMP-3 binding proteins of the present invention have an apparent affinity for TIMP-3 of 1000 pM or lower. In other embodiments, the TIMP-3 binding proteins exhibit an apparent affinity of 500 pM or lower, 200 pM or lower, 100 pM or lower, or 80 pM or lower. In another embodiment, the TIMP-3 binding protein exhibits an apparent affinity substantially the same as that of an antibody or LRP-peptide described herein in the Examples.

In another embodiment, the present invention provides a TIMP-3 binding protein that competes for binding to TIMP-3 with a TIMP-3 binding protein disclosed herein. Such competitive ability can be determined by methods that are well-known in the art, for example by competition in binding to TIMP-3 in an assay such as an ELISA, or by competition in another assay described herein. In one aspect, a TIMP-3 binding protein that competes for binding to TIMP-3 with a polypeptide disclosed herein binds the same epitope or an overlapping (or adjacent) epitope as the polypeptide. In another aspect, the TIMP-3 binding protein that competes for binding to TIMP-3 with a polypeptide disclosed herein agonizes an activity of TIMP-3.

In another aspect, the present invention provides a TIMP-3 binding protein having a half-life of at least one day in vitro or in vivo (e.g., when administered to a human subject). In one embodiment, the TIMP-3 binding protein has a half-life of at least three days. In another embodiment, the TIMP-3 binding protein has a half-life of four days or longer. In another embodiment, the TIMP-3 binding protein has a half-life of eight days or longer. In another embodiment, the TIMP-3 binding protein is derivatized or modified such that it has a longer half-life as compared to the underivatized or unmodified TIMP-3 binding protein. In another embodiment, the TIMP-3 binding protein contains one or more point mutations to increase serum half life, such as described in WO 00/09560, published Feb. 24, 2000, incorporated by reference.

In another aspect, the polypeptide (include antigen binding proteins) of the present invention comprises a derivative of a polypeptide. The derivatized polypeptide can comprise any molecule or substance that imparts a desired property to the polypeptide, such as increased half-life in a particular use. The derivatized polypeptide can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the polypeptide for a particular use (e.g., administration to a subject, such as a human subject, or other in vivo or in vitro uses). In one such example, the polypeptide is derivatized with a ligand that specifically bind to articular cartilage tissues, for example as disclosed in WO2008063291 and/or Rothenfluh et al., Nature Materials 7:248 (2008).

Examples of molecules that can be used to derivatize a polypeptide include albumin (e.g., human serum albumin) and polyethylene glycol (PEG). Albumin-linked and PEGylated derivatives of polypeptides can be prepared using techniques well known in the art. In one embodiment, the polypeptide is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols and polyvinyl alcohols (US Pat. App. No. 20030195154).

In another aspect, the present invention provides methods of screening for a molecule that binds to TIMP-3 using the TIMP-3 binding proteins of the present invention. Any suitable screening technique can be used. In one embodiment, a TIMP-3 molecule, or a fragment thereof to which a TIMP-3 binding protein of the present invention binds, is contacted with the TIMP-3 binding protein of the invention and with another molecule, wherein the other molecule binds to TIMP-3 if it reduces the binding of the TIMP-3 binding protein to TIMP-3. Binding of the TIMP-3 binding protein can be detected using any suitable method, e.g., an ELISA. Detection of binding of the TIMP-3 binding protein to TIMP-3 can be simplified by detectably labeling the TIMP-3 binding protein, as discussed above. In another embodiment, the TIMP-3-binding molecule is further analyzed to determine whether it agonizes a TIMP-3 activity (i.e., increases accumulation of TIMP-3 and/or enhances endogenous levels of TIMP-3).

### Compositions

Also comprehended by the invention are pharmaceutical compositions comprising effective amounts of polypeptide products of the invention together with pharmaceutically acceptable diluents, preservatives, solubilizers, emulsifiers, adjuvants and/or carriers useful in TIMP-3 therapy (i.e., conditions in which increasing the endogenous levels of TIMP-3 are useful). Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); covalent attachment of polymers such as polyethylene glycol to the protein (as discussed supra, see, for example U.S. Pat. No. 4,179,337 hereby incorporated by reference); incorporation of the material into particulate preparations of polymeric compounds such as polylactic acid, polyglycolic acid, etc. or into liposomes. Such compositions will influence the physical state, stability, rate of in vivo release, and rate of in vivo clearance of TIMP-3 binding proteins. See, e.g., Remington's Pharmaceutical Sciences, 18th Ed. (1990, Mack Publishing Co., Easton, Pa. 18042) pages 1435-1712 which are herein incorporated by reference.

Generally, an effective amount of the present polypeptides will be determined by the age, weight and condition or severity of disease of the recipient. See, Remingtons Pharmaceutical Sciences, supra, at pages 697-773, herein incorporated by reference. Typically, a dosage of between about 0.001 g/kg body weight to about 1 g/kg body weight, may be used, but more or less, as a skilled practitioner will recognize, may be used. For local (i.e., non-systemic) applications, such as topical or intra-articular applications, the dosing may be between about 0.001 g/cm² to about 1 g/cm². Dosing may be one or more times daily, or less frequently, and may be in conjunction with other compositions as described herein. It should be noted that the present invention is not limited to the dosages recited herein.

As is understood in the pertinent field, pharmaceutical compositions comprising the molecules of the invention are administered to a subject in a manner appropriate to the indication. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of an aerosol form, and the like. Other alternatives include eyedrops; oral preparations including pills, syrups, lozenges or chewing gum; and topical preparations such as lotions, gels, sprays, and ointments.

A plurality of agents act in concert in order to maintain the dynamic equilibrium of the extracellular matrix and tissues. In treatment of conditions where the equilibrium is skewed, one or more of the other agents may be used in conjunction with the present polypeptides. These other agents may be co-administered or administered in seriatim, or a combination thereof. Generally, these other agents may be selected from the list consisting of the metalloproteinases, serine proteases, inhibitors of matrix degrading enzymes, intracellular enzymes, cell adhesion modulators, and factors regulating the expression of extracellular matrix degrading proteinases and their inhibitors. While specific examples are listed below, one skilled in the art will recognize other agents performing equivalent functions, including additional agents, or other forms of the listed agents (such as those produced synthetically, via recombinant DNA techniques, and analogs and derivatives).

Other degradation inhibitors may also be used if increased or more specific prevention of extracellular matrix degradation is desired. Inhibitors may be selected from the group consisting of alpha₂ macroglobulin, pregnancy zone protein, ovostatin, alpha₁-proteinase inhibitor, alpha₂-antiplasmin, aprotinin, protease nexin-1, plasminogen activator inhibitor (PAI)-1, PAI-2, TIMP-1, and TIMP-2. Others may be used, as one skilled in the art will recognize.

Intracellular enzymes may also be used in conjunction with the present polypeptides. Intracellular enzymes also may affect extracellular matrix degradation, and include lysozomal enzymes, glycosidases and cathepsins.

Cell adhesion modulators may also be used in combination with the present polypeptides. For example, one may wish to modulate cell adhesion to the extracellular matrix prior to, during, or after inhibition of degradation of the extracellular matrix using the present polypeptides. Cells which have exhibited cell adhesion to the extracellular matrix include osteoclasts, macrophages, neutrophils, eosinophils, killer T cells and mast cells. Cell adhesion modulators include peptides containing an “RGD” motif or analog or mimetic antagonists or agonists.

Factors regulating expression of extracellular matrix degrading proteinases and their inhibitors include cytokines, such as IL-1 and TNF-alpha, TGF-beta, glucocorticoids, and retinoids. Other growth factors effecting cell proliferation and/or differentiation may also be used if the desired effect is to inhibit degradation of the extracellular matrix using the present polypeptides, in conjunction with such cellular effects. For example, during inflammation, one may desire the maintenance of the extracellular matrix (via inhibition of enzymatic activity) yet desire the production of neutrophils; therefore one may administer G-CSF. Other factors include erythropoietin, interleukin family members, SCF, M-CSF, IGF-I, IGF-II, EGF, FGF family members such as KGF, PDGF, and others. One may wish additionally the activity of interferons, such as interferon alpha's, beta's, gamma's, or consensus interferon. Intracellular agents include G-proteins, protein kinase C and inositol phosphatases. The use of the present polypeptides may provide therapeutic benefit with one or more agents involved in inflammation therapy.

Cell trafficking agents may also be used. For example, inflammation involves the degradation of the extracellular matrix, and the movement, or trafficking of cells to the site of injury. Prevention of degradation of the extracellular matrix may prevent such cell trafficking. Use of the present polypeptides in conjunction with agonists or antagonists of cell trafficking-modulation agents may therefore be desired in treating inflammation. Cell trafficking-modulating agents may be selected from the list consisting of endothelial cell surface receptors (such as E-selectins and integrins); leukocyte cell surface receptors (L-selectins); chemokins and chemoattractants. For a review of compositions involved in inflammation, see Carlos et al., Immunol. Rev. 114: 5-28 (1990), which is herein incorporated by reference.

Moreover, compositions may include neu differentiation factor, “NDF,” and methods of treatment may include the administration of NDF before, simultaneously with, or after the administration of TIMP-3. NDF has been found to stimulate the production of TIMP-2, and the combination of NDF, TIMP-1, -2 and/or -3 may provide benefits in treating tumors.

Polypeptide products of the invention may be “labeled” by association with a detectable marker substance (e.g., radiolabeled with ¹²⁵I) to provide reagents useful in detection and quantification of TIMP-3 in solid tissue and fluid samples such as blood or urine. Nucleic acid products of the invention may also be labeled with detectable markers (such as radiolabels and non-isotopic labels such as biotin) and employed in hybridization processes to identify relevant genes, for example.

The TIMP-3 binding compositions described herein modify the pathogenesis and provide a beneficial therapy for diseases or conditions characterized by matrix degradation and/or inflammation, i.e., those in which metalloproteinases play a deleterious role. The TIMP-3 binding compositions may be used alone or in conjunction with one or more agents used in treating such conditions. Accordingly, the present TIMP-3 binding compositions may be useful in the treatment of any disorder where excessive matrix loss is caused by metalloproteinase activity. The inventive TIMP-3 binding proteins are useful, alone or in combination with other drugs, in the treatment of various disorders linked to the overproduction of collagenase, aggrecanase, or other matrix-degrading or inflammation-promoting enzyme(s), including dystrophic epidermolysis bullosa, osteoarthritis, Reiter's syndrome, pseudogout, rheumatoid arthritis including juvenile rheumatoid arthritis, ankylosing spondylitis, scleroderma, periodontal disease, ulceration including corneal, epidermal, or gastric ulceration, wound healing after surgery, and restenosis. Other pathological conditions in which excessive collagen and/or proteoglycan degradation may play a role and thus where TIMP-3 binding proteins can be applied, include emphysema, Paget's disease of bone, osteoporosis, scleroderma, pressure atrophy of bone or tissues as in bedsores, cholesteatoma, and abnormal wound healing. TIMP-3 binding proteins can additionally be applied as an adjunct to other wound healing promoters, e.g., to modulate the turnover of collagen during the healing process.

Many metalloproteinases also exhibit pro-inflammatory activity; accordingly, additional embodiments include methods of treating inflammation and/or autoimmune disorders, wherein the disorders include, but are not limited to, cartilage inflammation, and/or bone degradation, arthritis, rheumatoid arthritis, pauciarticular rheumatoid arthritis, polyarticular rheumatoid arthritis, systemic onset rheumatoid arthritis, ankylosing spondylitis, enteropathic arthritis, reactive arthritis, Reiter's Syndrome, SEA Syndrome (Seronegativity, Enthesopathy, Arthropathy Syndrome), dermatomyositis, psoriatic arthritis, scleroderma, systemic lupus erythematosus, vasculitis, scleroderma, systemic lupus erythematosus, vasculitis, myolitis, polymyolitis, dermatomyolitis, osteoarthritis, polyarteritis nodossa, Wegener's granulomatosis, arteritis, polymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease, ulcerative colitis, Crohn's disease, Celiac disease (nontropical Sprue), enteropathy associated with seronegative arthropathies, microscopic or collagenous colitis, eosinophilic gastroenteritis, or pouchitis resulting after proctocolectomy and ileoanal anastomosis, pancreatitis, insulin-dependent diabetes mellitus, mastitis, cholecystitis, cholangitis, pericholangitis, multiple sclerosis (MS), asthma (including extrinsic and intrinsic asthma as well as related chronic inflammatory conditions, or hyperresponsiveness, of the airways), chronic obstructive pulmonary disease (COPD. i.e., chronic bronchitis, emphysema), Acute Respiratory Disorder Syndrome (ARDS), respiratory distress syndrome, cystic fibrosis, pulmonary hypertension, pulmonary vasoconstriction, acute lung injury, allergic bronchopulmonary aspergillosis, hypersensitivity pneumonia, eosinophilic pneumonia, bronchitis, allergic bronchitis bronchiectasis, tuberculosis, hypersensitivity pneumonitis, occupational asthma, asthma-like disorders, sarcoid, reactive airway disease (or dysfunction) syndrome, byssinosis, interstitial lung disease, hyper-eosinophilic syndrome, rhinitis, sinusitis, and parasitic lung disease, airway hyperresponsiveness associated with viral-induced conditions (for example, respiratory syncytial virus (RSV), parainfluenza virus (PIV), rhinovirus (RV) and adenovirus), Guillain-Barre disease, Graves' disease, Addison's disease, Raynaud's phenomenon, autoimmune hepatitis, GVHD, and the like.

TIMP-3 binding proteins also have application in cases where decreased relative levels of TIMP-3 (i.e., a decrease in the ratio of endogenous TIMP-3 to metalloproteases, which may be a result of decreased amounts of TIMP-3 or increased amounts of metalloproteases) are associated with pathological effects, for example, in myocardial ischemia, reperfusion injury, and during the progression to congestive heart failure.

Based on the ability of TIMP-3 to inhibit connective tissue degradation, agonizing TIMP-3 binding proteins have application in cases where inhibition of angiogenesis is useful, e.g., in preventing or retarding tumor development, and the prevention of the invasion of parasites. For example, in the field of tumor invasion and metastasis, the metastatic potential of some particular tumors correlates with the increased ability to synthesize and secrete collagenases, and with the inability to synthesize and secrete significant amounts of a metalloproteinase inhibitor. TIMP-3 binding proteins also have therapeutic application in inhibiting tumor cell dissemination during removal of primary tumors, during chemotherapy and radiation therapy, during harvesting of contaminated bone marrow, and during shunting of carcinomatous ascites. Diagnostically, correlation between absence of TIMP-3 production in a tumor specimen and its metastatic potential is useful as a prognostic indicator as well as an indicator for possible prevention therapy.

In addition, the present compositions and methods may be applicable for cosmetic purposes, in that localized inhibition of connective tissue breakdown may alter the appearance of tissue.

MMPs also act on the basal lamina and tight junction proteins in the brain, as part of the pathway for opening the blood-brain barrier (BBB), facilitating the entrance of cells and soluble mediators of inflammation into the brain. Accordingly, the present compositions and methods may be useful in the treatment of disorders of the nervous system characterized by excessive or inappropriate permeabilization of the BBB. Additionally, degradation of matrix proteins around neurons can result in loss of contact and cell death; thus, TIMP-3 binding compositions may protect nerve cells from damage by preserving the basement membrane surrounding nerve cells. The inventive TIMP-3 binding compositions are useful in treating or ameliorating the neuroinflammatory response to injury, for example, cerebral ischemia. The compositions disclosed herein will also be useful in the treatment of neurodegenerative diseases where inflammation is an underlying cause of the disease, for example, multiple sclerosis, as well as in treatment of various forms of neuropathy and/or myopathy, spinal cord injury, and arnyotrophic lateral sclerosis (ALS) Accordingly, uses of the inventive compositions may involve co-administration with BDNF, NT-3, NGF, CNTF, NDF, SCF, or other nerve cell growth or proliferation modulation factors.

As described above, the present TIMP-3 binding proteins have wide application in a variety of disorders. Thus, another embodiment contemplated herein is a kit including the present polypeptides and optionally one or more of the additional compositions described above for the treatment of a disorder involving the degradation of extracellular matrix. An additional embodiment is an article of manufacture comprising a packaging material and a pharmaceutical agent within said packaging material, wherein said pharmaceutical agent contains the present polypeptide(s) and wherein said packaging material comprises a label which indicates that said pharmaceutical agent may be used for an indication selected from the group consisting of: cancer, inflammation, arthritis (including osteoarthritis and the like), dystrophic epidermolysis bullosa, periodontal disease, ulceration, emphysema, bone disorders, scleroderma, wound healing, erythrocyte deficiencies, cosmetic tissue reconstruction, fertilization or embryo implant modulation, and nerve cell disorders. This article of manufacture may optionally include other compositions or label descriptions of other compositions.

The following examples are provided for the purpose of illustrating specific embodiments or features of the instant invention and do not limit its scope.

### Example 1

This example describes the internalization of exogenous TIMP-3 as examined by confocal microscopy. A549 cells (a continuous tumor-cell line from a human lung carcinoma with properties of type II alveolar epithelial cells, either wild-type or A549 cells lacking the LRP-1 gene) were incubated typically with 1 microG/ml TIMP-3 for 30 minutes at 4 degrees, with or without various pre-treatments (including heparin), then washed, fixed and stained with a fluorescently labeled anti-TIMP-3 antibody, or washed and then incubated further at 4 or 37 degrees before fixation and staining. Cells were also assayed for the accumulation of TIMP-3 in the culture medium, by Western blot or ELISA, substantially as described herein.

Cells that lack LRP-1 were found to accumulate TIMP-3 in the medium to a greater extent than wild-type cells. The confocal microscopic analysis showed that TIMP-3 binds to the cell surface, rapidly disappears when the cells are incubated at 37 degrees but not at 4 degrees, and accumulates inside the cells if they are pre-treated with chloroquine to prevent lysosomal degradation. Moreover, the binding of TIMP-3 to the cell surface was decreased in the presence of heparin.

### Example 2

This example describes the preparation and purification of LRP-1 peptides. Various peptides from the ecto-domain of LRP-1 were expressed in E. coli, purified by as described below and tested for binding to TIMP-3 by both plate- and bead-based binding assays. The amino acid sequence of LRP-1 is shown in SEQ ID NO:1; Table 1 below lists the various peptides that were expressed, referring to the amino acid sequence of LRP-1. The peptides are referred to herein as monomers (for example, LA3, LA4, LA5, and other peptides designated by a single number) or multimers (for example, LA3-5, LA5-7, LA8-10, and other peptides designated by multiple numbers).

TABLE 1
LRP-1 Peptides

| Peptide | Amino |
|---|---|
| designation | acids |
| Cluster I (C I) | 27-114 |
| Cluster II (C II) | 854-1184 |
| Cluster III (C III) | 2524- |
| Cluster IV (C IV) | 3334- |
| LA3-5 | 854-975 |
| LA5-7 | 931-1061 |
| LA8-10 | 1062- |
| LA11-13 | 2524- |
| LA13-15 | 2605- |
| LA15-17 | 2696- |
| LA18-20 | 2818- |
| LA21-23 | 3334- |
| LA24-26 | 3453- |
| LA26-28 | 3536- |
| LA29-31 | 3654- |
| LA4 | 895-930 |
| LA5 | 931-975 |
| LA6 | 976-1014 |
| LA7 | 1015- |
| LA15 | 2696- |
| LA16 | 2734-2773 |
| LA17 | 2774-2817 |
| LA18 | 2818-2857 |
| LA19 | 2858-2903 |
| LA20 | 2904-2943 |
| LA21 | 3334-3373 |
| LA22 | 3374-3412 |
| LA23 | 3413-3452 |
| LA24 | 3453-3493 |
| LA25 | 3494-3535 |
| LA26 | 3536-3574 |
| LA27 | 3575-3612 |
| LA28 | 3613-3653 |
| LA3-4 | 854-930 |
| LA4-5 | 895-975 |
| LA5-6 | 931-1014 |
| LA23-24 | 3413-3493 |
| LA24-25 | 3453-3535 |
| LA25-26 | 3594-3574 |
| LA3 | 854-894 |

LRP-1 peptides are prepared using standard recombinant protein techniques in E. coli bacteria. A culture is inoculated from a single transformed E. coli colony for each peptide of interest, into 3 mL of 2xYT medium (a nutritionally rich growth medium for recombinant strains of E. coli) containing 40 microG/mL Kanamycin and grown to saturation, overnight at 300 rpm at 37° C. The next morning, 1.8 mL of overnight culture is inoculated into 500 mL shake flask of 2xYT containing 40 microG/mL Kanamycin and grown with shaking at 300 rpm at 37° C. until OD₆₀₀ is between 0.8 and 1.0 (about 3 hrs). Cultures are induced with 500 microL of 1 M IPTG (Isopropyl-beta-D-thiogalactoside; 1 mM final) and growth continued with shaking at 300 rpm at 37° C. for 3 hrs. Cultures are then transferred to 500 mL Nalgene bottles (Thermo Fisher Scientific, Rochester, N.Y.) and centrifuged for 12 minutes at 8000 rpm to pellet cells. Supernatant is removed and pellets are resuspended in 20 mL of Equilibrate/Bind/Wash buffer (20 mM Tris pH 7.5, 20 mM Imidazole, 1 mM CaCl₂, 300 mM NaCl). Resuspended cells are transferred into a 30 mL Oak Ridge centrifuge tube (VWR, West Chester, Pa.) and lysed by heating in an 80° C. water bath for 15 minutes. Lysed cells are cooled on ice water for about 10 minutes then centrifuged for 30 minutes at 18,000 rpm at 4° C.

Prepare enough Ni-NTA agarose (Qiagen, Valencia, Calif.) for 1.5 mL agarose per peptide (3 mL total volume including buffer) by washing agarose three times with Equilibrate/Bind/Wash Buffer to remove ethanol. After the third wash, Ni-NTA agarose is resuspended to original volume with same buffer. Next, three mLs agarose/buffer mixture is added to 50 mL flat top, screw cap polypropylene tubes (Falcon', available from BD Biosciences, San Jose Calif.). After pelleting lysed cells, each protein supernatant is removed and added to a tube containing 3 mL washed Ni-NTA agarose. Protein is allowed to bind to Ni-NTA agarose for 0.5 hour at room temperature with rocking. Then the Ni-NTA resin plus bound protein is transferred to disposable gravity columns (Clontech, Mountain View, Calif.) mounted to a vacuum manifold (Qiagen). Next, the Ni-NTA resin with bound protein is washed with at least 30 column volumes of Equilibrate/Bind/Wash buffer, without allowing resin to run dry while washing. Columns containing washed resin are then placed on top of clean 15 mL polypropylene collection tubes (Falcon') and protein is eluted with 2 mL times 2 (4 mL total) of Ni-NTA Elution buffer (20 mM Tris pH 7.5, 200 mM Imidazole, 1 mM CaCl₂, 300 mM NaCl). Eluted proteins are then dialyzed into buffer containing redox reagents [20 mM Tris pH 7.5, 50 mM NaCl, 1 mM CaCl₂] plus 1 mM 2-Mercaptoethanol (Sigma-Aldrich, St. Louis, Mo.) and 250 microM 2-Hydroxyethyl disulfide (Sigma-Aldrich) overnight at 4° C. The next day, proteins are dialyzed into buffer without redox reagents (20 mM Tris pH 7.5, 50 mM NaCl, 1 mM CaCl₂) for 3 hrs. at 4° C. This is then repeated. Proteins are then filtered using 0.2 micron filter (Pall Corporation, East Hills N.Y.) and stored at 4° C. until next purification step.

For the next step, 1.3 mL slurry of Q-Sepharose™ (an ion exchange chromatogroahpy resin with a quaternary ammonium strong anion; ˜1 mL resin; GE Healthcare, Piscataway, N.J.) per peptide is added to 15 mL disposable gravity columns (Clontech). Columns are equilibrated with 10 mL times 2 of Equilibration Buffer (20 mM Tris pH 7.5, 1 mM CaCl₂, 50 mM NaCl) and allowed to drain by gravity. Next, ˜3.9 mL of Ni-NTA purified protein is added gently to the Q-Sepharose™ resin and flow thru is collected into 15 mL polyprolylene tubes (BD Falcon™, BD Biosciences, San Jose, Calif.). Resin is washed with 5 mL times 5 of Wash Buffer (20 mM Tris pH 7.5, 1 mM CaCl₂, 50 mM NaCl). The protein is eluted off the resin with a NaCl salt gradient used for monomer or ultimers forms of the LRP-1 peptides (see below) and collected into 96-well 2 mL polypropylene plates. Elution is done with 1.3 mL×2 fractions per protein for each NaCl concentration. The NaCl gradient for monomer forms was: [80 mM, 110 mM, 150 mM, 180 mM, 200 mM, 250 mM] and for ultimers forms was: [100 mM, 150 mM, 180 mM, 220 mM, 250 mM, 300 mM]. Once protein is eluted, a Bradford assay is run in order to select fractions containing protein. A gel is run of selected purification fractions-5 microL/well of Ni-NTA purified load protein and 10 microL/well of eluted Q-Sepharose™ fractions. Fractions containing protein are selected, pooled, and stored at 4° C. (short term) or −80° C. (long term).

### Example 3

This example describes the binding of LRP-1 peptides to recombinant human and mouse TIMP-3 as evaluated using enzyme-linked immunosorbent assays (ELISA) and AlphaScreen®.

### Directly Coated TIMP-3 Binding ELISA:

A MaxiSorp™ plate (a 96-well polystyrene plate with high affinity to molecules with mixed hydrophilic/hydrophobic domains; Nunc Thermo Fisher Scientific, Roskilde, Denmark,) is coated with 100 microL/well of 100 nM human or mouse TIMP-3 diluted in coating buffer (TBS [pH 7.5], 1 mM CaCl₂) and incubated overnight at 4 C. After incubation, the coating solution is removed and replaced with 250 microL/well of blocking buffer (1% BSA, TBS [pH 7.5], 1 mM CaCl₂) and incubated with shaking for 1 hour at room temperature. The plates are then washed three times with 200 microL/well with wash buffer 1 (TBS [pH 7.5], 1 mM CaCl₂). The LRP-1 peptides are titrated in assay buffer (0.1% BSA, TBS [pH 7.5], 1 mM CaCl₂, 0.02% Tween-20) in a separate polypropylene non-treated round-bottom 96-well plate (BD Falcon™) starting at 1 microM concentration and then diluted serially 4-fold for 8-points, with the last point buffer only. Then 100 microL/well of the diluted LRP-1 peptide titration is added to the TIMP-3 coated plate and allowed to bind for 1.5 hour at room temperature with shaking. After incubation, the plate is washed three times with 200 microL/well of wash buffer 2 (TBS [pH 7.5], 1 mM CaCl₂, 0.02% Tween-20). A 1:5 K dilution of 100 microG/mL rat-anti-HA (clone3F10)-HRP (a high affinity rat monoclonal antibody that recognizes a peptide sequence derived from the influenza hemagglutinin protein, conjugated to horseradish peroxidase; Roche Diagnostics, Indianapolis, Ind.) is added at 100 microL/well diluted in assay buffer and incubated for 1 hr. at room temperature on plate shaker. The assay plate is then washed three times with 200 microL/well with wash buffer 2. The plate is developed by adding 100 microL/well of 1:1 mixture of TMB and H₂O₂ (Pierce, Fisher Thermo Scientific, Rockford Il.), the reaction is stopped by adding 100 microL/well 2NH₂SO₄ (VWR), and read on a SpectraMax microplate reader (Molecular Devices, Sunnyvale, Calif.) at OD450 nm using SOFTmax Pro software version 3.1.2. Data is analyzed using GraphPad Prism 4.01 software. Binding of LRP-1 peptides to TIMP-3 is expressed as binding EC50.

Indirect Binding ELISA with TIMP-3 Presented Via Anti-TIMP-3 Neutralizing Antibody:

A MaxiSorp™ plate is coated with 100 microL/well of 10 nM mouse anti-human TIMP-3 antibody (R & D Systems, Minneapolis, Minn., neutralizing antibody clone 277128, which does not bind mouse TIMP-3) diluted in coating buffer (TBS [pH 7.5]/1 mM CaCl₂) and incubated overnight at 4 C. After incubation, the plate contents are discarded and 250 microL/well of blocking buffer (1% BSA, TBS [pH 7.5], 1 mM CaCl₂) is added to the plate which is incubated with shaking for 1 hour at room temperature. The plate is then washed three times with 200 microL/well with wash buffer 1 (TBS [pH 7.5], 1 mM CaCl₂) followed by the addition of 100 microL of 20 nM recombinant human TIMP-3. The plate is incubated at room temperature for 1 hour. The LRP-1 peptides are titrated as described previously, and 100 microL/well of the diluted LRP-1 peptide titration is added to the TIMP-3 coated plate and allowed to bind for 1.5 hour at room temperature with shaking. After incubation, the plate is washed and the ELISA performed substantially as described previously for the direct binding ELISA.

### TIMP-3 AlphaScreen Binding Assay:

Peptides were also evaluated by AlphaScreen® (Amplified Luminescent Proximity Homogeneous Assay; PerkinElmer, Waltham, Mass.) a very sensitive non-radioactive homogeneous assay technology that allows the screening of a large range of biological interactions and activities, substantially according to the manufacturer's instructions. Briefly, all dilutions are made in AlphaScreen® Buffer: [40 mM HEPES pH 7.5, 100 mM NaCl, 1 mM CaCl2, 0.1% BSA, 0.05% Tween-20]. The LRP-1 peptides are titrated in a separate polypropylene 96-well plate (Falcon) starting at 4 microM concentration and then diluted serially 3-fold for 12-points, with the last point buffer only. First 2 microL/well of protein titration curve is added to white, small volume, 384-well assay plate (Greiner Bio-One, Stonehouse, UK) in duplicate. Next, 2 microL of biotinylated (AFS, in-house conjmicroGation) recombinant human or mouse TIMP-3, is added at 12 nM (or 3 nM final assay concentration) to the assay plate. Lastly, in subdued light 4 microL of a mixture of streptavidin donor beads, anti-mouse IgG acceptor beads (PerkinElmer) both diluted to 20 microG/mL (10 microG/mL final assay concentration) and 2 nM (1 nM final concentration) rat-anti-HA (clone3F10)-HRP (Roche Diagnostics) is added to the plates. Plates containing 8 microL final assay volme are covered with TopSeal A (to prevent evaporation), spun quickly at 1000 rpm, and incubated overnight (covered with foil to prevent light exposure) before reading on Fusion plate reader (PerkinElmer) using AlphaScreen® parameters (excitation 680 nM and emission 520-620 nm). Data is analyzed using GraphPad Prism 4.01 software. Binding to TIMP-3 is reported as total signal (cps). Results of several experiments are shown in Table 2-3 below.

TABLE 2
Binding of LRP-1 peptides by ELISA and/or AlphaScreen ®

| | Binding ELISA | | | AlphaScreen ® | |
|---|---|---|---|---|---|
| | EC50 (nm) | | | total signal (cps) | |
| | Assay 1 | Assay 2 | Assay 3 | Assay 1 | Assay 2 |
| LA3-10 | 45.5 | ND | 32.5 | ND | 100,000 |
| LA3-5 | 52.5 | ND | 214 | 4,000 | 9,000 |
| LA5-7 | 210 | ND | 350 | 2,000 | 8,000 |
| LA8-10 | 1282/276 ¹ | ND | 657/1042 ¹ | 110,000 | 100,000 |
| LA3-4 | ND | 289 | 500 | 9,000 | 3,500 |
| LA4-5 | ND | ~2000 | ND | 2,200 | 2,000 |
| LA5-6 | ND | 400 | 512 | 6,000 | 4,000 |
| LA8-9 | ND | ND | 249 | ND | 6,000 |
| LA9-10 | ND | ND | ~3000 | ND | 2,000 |
| LA3 | ND | +/− | ND | 2,200 | 2,000 |
| LA4 | ND | 900 | ~3000 | 2,200 | 2,000 |
| LA5 | ND | 1084 | ND | 2,200 | 2,000 |
| LA6 | ND | 1214 | ND | 2,200 | ND |
| LA7 | ND | ~2500 | ND | 2,200 | ND |
| LA8 | ND | ND | 396 | ND | 2,000 |
| LA9 | ND | ND | ~1500 | ND | 2,000 |
| LA10 | ND | ND | >3000 | ND | 3,000 |
| LA11-20 | ND | ND | ND | ND | ND |
| LA11-13 | ~5000 | ND | ND | 1,200 | ND |
| LA13-15 | NS | ND | ND | 1,200 | ND |
| LA15-17 | 193 | ND | 191 | 90,000 | 70,000 |
| LA18-20 | ~300 | ND | ND | 3,000 | ND |
| LA15 | ND | ~5000 | ~1500 | 2,200 | 2,000 |
| LA16 | ND | — | ND | 2,200 | ND |
| LA17 | ND | >5000 | ND | 2,200 | ND |
| LA18 | ND | >10000 | ND | 2,200 | ND |
| LA19 | ND | >10000 | ND | 2,200 | ND |
| LA20 | ND | >10000 | ND | 2,200 | ND ND |
| LA21-31 | 92 ² | ND | ND | 30,000 | ND |
| LA21-23 | 243 | ND | ND | 3,000 | |
| LA24-26 | 46/26 ¹ | 80 | 68 | 150,000 | 120,000 |
| LA26-28 | 268 | ND | ND | 6,500 | 9,000 |
| LA29-31 | ~1000/~300 ¹ | ND | ND | 1,200 | ND |
| LA23-24 | ND | ~600 | ND | 2,200 | 2,000 |
| LA24-25 | ND | 211 | 436 | 290,000 | 130,000 |
| LA25-26 | ND | 673 | ND | 20,000 | 5,000 |
| LA21 | ND | 1019 | ND | 2,200 | ND |
| LA22 | ND | 426 | ND | 2,200 | ND |
| LA23 | ND | >5000 | ND | 2,200 | ND |
| LA24 | ND | ~500 ³ | ~1000 | 2,200 | 2,000 |
| LA25 | ND | 1217 | ~1000 | 5,000 | 2,000 |
| LA26 | ND | 300 | ND | 2,200 | ND |
| LA27 | ND | — | ND | 2,200 | ND |
| LA28 | ND | 300 ² | ND | 2,200 | ND |

ND: Not Done

TABLE 3
EC50 of LRP-1 Peptides in TIMP-3 Binding ELISA

| | Indirect | Direct | Direct |
|---|---|---|---|
| | HuTIMP-3 | HuTIMP-3 | MuTIMP-3 |
| LA3-5 | 8 | 78 | 71 |
| LA5-7 | 9 | 94 | 74 |
| LA8-10 | 219 | >1000 | 696 |
| LA3-4 | 81 | 226 | 155 |
| LA5-6 | 78 | >1000 | 332 |
| LA8-9 | 113 | 181 | 173 |
| LA4 | no binding | no binding | no binding |
| LA6 | 9 | 95 | 38 |
| LA7 | >1000 | >1000 | >1000 |
| LA8 | 198 | 292 | 255 |
| LA15-17 | 100 | 510 | 306 |
| LA15 | 378 | 901 | >1000 |
| LA17 | >1000 | 886 | >1000 |
| LA24-26 | 8 | 28 | 15 |
| LA23-24 | 85 | 187 | 140 |
| LA24-25 | 32 | 162 | 124 |
| LA25-26 | 41 | 167 | 117 |
| LA25-26 | 20 | 99 | 80 |
| LA21 | 313 | 303 | 206 |
| LA22 | 185 | 456 | 394 |
| LA22 | 200 | 340 | 478 |
| LA24 | 10 | 68 | 66 |
| LA25 | 447 | 742 | >1000 |
| LA26 | 179 | 118 | 134 |
| LA28 | 972 | >1000 | >1000 |

Multiple ecto-domain LRP-1 peptides bound TIMP-3 with submicromolar affinity, including Cluster II (which contains 8 ligand binding domains) and parts of Cluster IV. Peptides that bound TIMP-3 were tested for interference with TIMP-3's inhibition of MMP-13, using a fluorescence-quench substrate, and for promotion of TIMP-3 accumulation in HTB-94 cell cultures, as described below.

### Example 4

This Example described MMP-13 Inhibition Assays useful for measuring the effect(s) of TIMP-3 binding proteins (also referred to as test molecules, including LRP-1 peptides and antibodies to TIMP-3) on the ability of TIMP-3 to inhibit MMP-13. First, a titration of TIMP-3 inhibition of MMP-13 is run to empirically determine the IC₅₀ of TIMP-3. Typically the IC₅₀ of TIMP-3 to MMP-13 is 0.5 nM to 1 nM and is used to select the concentration used to characterize the test molecules. Briefly, test molecules are titrated in assay buffer and added to black polystyrene 96 or 384 well assay plate (Griener Bio-One, Germany). The concentrations of the test molecules are dependant on the activity of the molecule; for example, titrations may begin at 1000, 2000 or 3000 nM and use five-fold dilutions for titration, although other types of titrations may be used or a single concentration may be tested. Then recombinant TIMP-3 is diluted in assay buffer (20 mM Tris, 10 mM CaCl₂, 10 uM ZnCl₂, 0.01% Brij 35 (Calbiochem/EMD, San Diego, Calif.), pH 7.5) to a previously determined concentration (for example, TIMP-3's IC₇₀ with 3 nM being a typical concentration), added to the test molecules and rotated for 10 minutes at room temperature. Active MMP-13 (Calbiochem/EMD,) is diluted in assay buffer to give a final assay concentration of 1.46 nM added to the test molecule titration/TIMP-3 mixture and incubated for 10 minutes at room temperature in a final volume of 50 microL. Alternatively, pro-MMP-13 (R & D Systems, Minneapolis, Minn.) is activated with aminophenyl mercuric acetate (APMA; Calbiochem/EMD,) for 2 hours at 37 degrees C., and used in the assay.

A fluorogenic substrate such as Mca-PLGL-Dpa-AR-NH2 Fluorogenic MMP Substrate or Mca-KPLGL-Dpa-AR-NH2 Fluorogenic Peptide Substrate (R & D Systems) is prepared to a final assay concentration of 20 microM in assay buffer, and added to the MMP-13 enzyme/huTIMP-3/test molecule solution. MMP-13 activity is measured kinetically for 20 minutes using Molecular Devices fluorescent plate reader. The effect of the molecules being tested is expressed as percent of expected maximum TIMP-3 inhibition of MMP-13 enzymantic activity.

Test molecules are also analyzed for direct inhibition of MMP-13 activity in an assay substantially similar to that described above but in the absence of TIMP-3. Titrations of the test molecules in assay buffer may begin at 1000 nM and use five-fold dilutions for titration, although other types of titrations may be used or a single concentration may be tested. Active MMP-13 is diluted in the assay buffer to give a final assay concentration of 1.46 nM, added to the test molecule titration and incubated for 10 minutes at room temperature in a final volume of 50 microL. A fluorogenic substrate is prepared to a final assay concentration of 20 microM in assay buffer, and added to the MMP-13 enzyme/test molecule solution. MMP-13 activity is measured kinetically for 20 minutes using Molecular Devices fluorescent plate reader. The effect of the molecules tested is expressed as percent decrease of the MMP-13 enzymatic activity.

Table 4 below present the results of a set of experiments comparing the effect of various LRP-1 peptides on TIMP-3 Inhibition of MMP-13, as well as their direct effect on MMP13 activity. Peptides that demonstrated greater than 90% of the inhibition that occurred with TIMP-3 alone were viewed as not significantly affecting the ability of TIMP-3 to inhibit MMP-13; peptides that decreased the ability of TIMP-3 to inhibit MMP-13 by 10% or more (i.e., those that yielded 90% or less of the inhibitory activity observed with TIMP-3 alone) were viewed as having a negative effect on the inhibitory ability of TIMP-3. Peptides that by themselves decreased the activity of MMP-13 by 10% or more were viewed as significantly affecting MMP-13 activity.

TABLE 4
Effect of LRP-1 peptides on TIMP-3 Inhibition
of MMP-13 and/or on MMP13activity

| | Effect of LRP-1 peptides on the inhibition of | | | | Direct |
|---|---|---|---|---|---|
| | MMP13 by TIMP-3 (% of the inhibitory | | | | Inhibition |
| | activity of of TIMP-3 + peptide compared to | | | | of MMP13 |
| | TIMP-3 alone) | | | | (% decrease) |
| Peptide | 2000 nM | 400 nM | 80 nM | 16 nM | 1000 nM |
| LA3-5 | 101 | 103 | 101 | 104 | 2 |
| LA5-7 | 68 | 93 | 99 | 101 | 2 |
| LA8-10 | 93 | 84 | 89 | 94 | −6 |
| LA3-4 | 91 | 84 | 102 | 104 | ND |
| LA4-5 | 104 | 102 | 101 | 102 | 26 |
| LA5-6 | 65 | 72 | 80 | 97 | 16 |
| LA8-9 | 97 | 91 | 91 | 95 | 23 |
| LA9-10 | 107 | 105 | 107 | 105 | 34 |
| LA3 | 109 | 80 | 84 | 103 | 7 |
| LA4 | ND | ND | ND | ND | ND |
| LA5 | ND | ND | ND | ND | 69 |
| LA6 | ND | ND | ND | ND | 57 |
| LA7 | ND | ND | ND | ND | 49 |
| LA8 | 107 | 74 | 71 | 101 | 49 |
| LA9 | 107 | 105 | 104 | 104 | 41 |
| LA10 | 103 | 102 | 106 | 106 | 49 |
| LA11-20 | ND | ND | ND | ND | 26 |
| LA11-13 | ND | ND | ND | ND | −2 |
| LA13-15 | ND | ND | ND | ND | ND |
| LA15-17 | 91 | 83 | 82 | 95 | ND |
| LA18-20 | ND | ND | ND | ND | ND |
| LA15 | 58 | 67 | 95 | 104 | 61 |
| LA16 | 89 | 52 | 101 | 105 | 20 |
| LA17 | 77 | 77 | 93 | 103 | 41 |
| LA18 | ND | ND | ND | ND | ND |
| LA19 | ND | ND | ND | ND | ND |
| LA20 | ND | ND | ND | ND | 0 |
| LA21-31 | ND | ND | ND | ND | 29 |
| LA21-23 | 107 | 100 | 96 | 97 | 19 |
| LA24-26 | 103 | 105 | 95 | 92 | 59 |
| LA26-28 | 103 | 104 | 100 | 100 | 53 |
| LA29-31 | 96 | 97 | 99 | 96 | 5 |
| LA23-24 | 53 | 92 | 103 | 106 | ND |
| LA24-25 | 92 | 87 | 88 | 90 | ND |
| LA25-26 | 101 | 105 | 101 | 97 | ND |
| LA21 | ND | ND | ND | ND | −2 |
| LA22 | ND | ND | ND | ND | 59 |
| LA23 | 84 | 106 | 107 | 104 | 46 |
| LA24 | 81 | 103 | 104 | 104 | −22 |
| LA25 | 94 | 94 | 95 | 96 | ND |
| LA26 | 108 | 112 | 109 | 96 | 1 |
| LA27 | ND | ND | ND | ND | 47 |
| LA28 | ND | ND | ND | ND | 32 |

ND: Not done
Note:
certain peptides appeared to increase MMP-13 activity; these are shown with a negative number in the % decrease column.

The Cluster IV sequences substantially impaired TIMP-3's inhibition of MMP-13, but Cluster II had a minimal effect on this inhibitory activity.

### Example 5

This example describes an assay to evaluate the accumulation of TIMP-3 in the medium of cultured cells. HTB-94™ cells (a chondrocytic cell line available from the American Type Culture Collection, Manassas, Va.) are plated to 24-well tissue culture plates at 1×10̂5 cells per well in 1 ml growth medium (RPMI1640, 10% FBS, 1% PSG), and incubated at 37 C, 5% CO2 overnight. The cells are then washed once with PBS, and the wells are replenished with 250 microL serum-free medium (RPMI1640, 1% PSG; designated SF in the tables below) to which is added the polypeptide being tested or a control (for example medium containing heparin, 1 mg/ml). Proteins to be tested are diluted to appropriate concentrations, for example as shown in the Tables below, and added to the cells. Cells are then incubated at 37 C, 5% CO2 for two additional days, day 2 conditioned medium (CM) samples are collected and clarified by centrifugation (i.e., for 5 minutes at 10K RPM) and the supernatant fluid is collected and stored at −20 C until assayed. Analysis is done on 100 microL CM samples, either by Western blot or by using a standard human TIMP-3 ELISA as per the manufacturer's protocol (R&D Systems Inc., Minneapolis, Minn.).

Analysis by Western blot indicated that polypeptides from Cluster II (C II) of LRP-1 enhanced accumulation of TIMP-3 in conditioned medium from HTB-94™ cells, Cluster I, Cluster III and Cluster IV polypeptides did not result in significant accumulation. CM were also tested by ELISA, and approximate concentrations of TIMP-3 determined by comparing values to a standard curve; results of several such experiments are shown in Tables 5 through 7 below.

TABLE 5
Accumulation of TIMP3 in the presence of LRP-1
Cluster I and Cluster II domain polypeptides

| | | TIMP-3 (ng/ml) | |
|---|---|---|---|
| SF | 0.61 | na | na |
| Heparin | 10.98 | na | na |
| Polypeptide | 2 microM | 1 microM | 0.5 microM |
| C I | 0.81 | 0.86 | 1.02 |
| C II (prep 1) | 5.18 | 2.23 | 1.99 |
| C II (prep 2) | 1.92 | 1.71 | 1.35 |

TABLE 6
Accumulation of TIMP-3 in the presence
of LRP-1 peptides (2 microM)

| | TIMP-3 |
|---|---|
| | (ng/ml) |
| Hep | 14.55 |
| SF | 1.41 |
| C I | 0.65 |
| C II | 8.49 |
| LA3 | 0.60 |
| LA4 | 1.07 |
| LA5 | 0.94 |
| LA6 | 1.07 |
| LA7 | 0.90 |
| LA8 | 0.51 |
| LA9 | 0.63 |
| LA10 | 1.22 |
| LA15 | 1.29 |
| LA16 | 0.94 |
| LA17 | 1.06 |
| LA18 | 1.11 |
| LA19 | 1.06 |
| LA20 | 0.96 |
| LA21 | 1.82 |
| LA22 | 0.96 |
| LA23 | 0.87 |
| LA24 | 0.41 |

TABLE 7
Accumulation of TIMP-3 in the presence
of LRP-1 peptides (2 microM)

| | TIMP-3 |
|---|---|
| | (ng/ml) |
| Hep | 26.15 |
| SF | 1.71 |
| C I | 0.87 |
| C II | 13.42 |
| LA3-5 | 4.22 |
| LA8-10 | 3.91 |
| LA15-17 | 4.70 |
| LA21-23 | 4.46 |
| LA24-26 | 10.38 |
| LA26-28 | 1.57 |
| LA29-31 | 1.26 |
| LA4-5 | 2.68 |
| LA5-6 | 2.38 |
| LA8-9 | 3.46 |
| LA9-10 | 2.26 |
| LA23-24 | 1.38 |
| LA24-25 | 2.48 |
| LA25-26 | 9.30 |
| LA25 | 1.24 |
| LA26 | 1.01 |
| LA27 | 0.65 |
| LA28 | 0.99 |

Several Cluster II peptides resulted in an accumulation of TIMP-3 in HTB-94 cell cultures comparable to that seen in the presence of heparin, which is believed to prevent TIMP-3 from binding to the cell surface.

### Example 6

### Preparation of Monoclonal Antibodies

Fully human antibodies to TIMP-3 were generated by immunizing XenoMouse™ transgenic mice, strains used included XMG2-KL and XMG4-KL, (Mendez M J et al., Nat Gen, 1997, and Kellerman et al., Curr Opin Biotech 2002). Mice were immunized with soluble TIMP3-His protein for a sufficient time and with sufficient immunizations to exhibit specific immunoresponses (i.e., about 2.5 months total with biweekly boosting in the first four weeks using alternating injection routes, intra-peritoneal injections into the abdomen or sub-cutaneous injections at the base of the tail, followed with once weekly boosting for the last six weeks using the same alternating injection routes). Serum titer was monitored by enzyme-linked immunosorbent assay (ELISA) utilizing TIMP-3pHis-coated plates. Mice that exhibited specific anti-TIMP-3 immune responses were sacrificed and used for antibody generation.

Antibodies which specifically bind to TIMP3 were identified using an ELISA binding screen. For this assay, 384 well ELISA plates are coated, with 10 microG/ml neutravadin (a deglycosylated form of streptavidin, available from Pierce Fisher Thermo Scientific), overnight at 4° C. Plates are then loaded with 1 microG/ml of biotinylated TIMP3pHis. Supernatants identified as positive for binding to TIMP-3 (86 positive binding supernatants from the TIMP-3-polyHis immunization campaign and 33 positive binding supernatants from the TIMP-3pHis/KLH immunization campaign) were evaluated and ranked in several different assays, including interference with the inhibition of MMP13 by TIMP-3, relative quantitation of specific IgG concentration, and relative affinity to TIMP3 in a low-antigen setting. Antibodies were also evaluated for their effect on the accumulation of TIMP-3 in culture supernatant fluid substantially as described in Example 5; results are shown below.

TABLE 8
Accumulation of TIMP3 in the presence
of TIMP-3-specific antibodies

| | | TIMP-3 (ng/ml) | |
|---|---|---|---|
| Polypeptide | 2 microM | 1 microM | 0.5 microM |
| IgG1 | 4.42 | 2.09 | 1.76 |
| IgG2 | 3.99 | 2.70 | 2.52 |
| IgG4 | 3.48 | 2.73 | 2.02 |
| Cluster I | 1.44 | 1.19 | 1.41 |
| Cluster II | 2.76 | 1.94 | 1.59 |
| 3F3 | 3.76 | 2.11 | 1.92 |
| 10A7 | 7.96 | 5.89 | 4.90 |
| 8F1 | 9.79 | 6.39 | 5.66 |
| 4E4 | 3.00 | 1.10 | 0.78 |
| 8C5 | 7.76 | 4.70 | 4.02 |
| 11E11 | 2.12 | 1.04 | 1.68 |
| SF | 0.84 | na | na |
| Heparin | 17.31 | na | na |

When tested for interference with the ability of TIMP-3 to inhibit MMP-13 using an assay similar to that described previously, antibody 10A7 exhibited relatively little interference while the remaining antibodies exhibited greater interference. Antibodies 8F1, 8C5 and 10A7 also inhibited TIMP3 internalization as observed by confocal microscopy substantially as described previously.

### Example 7

### Preparation of Monoclonal Antibodies

Additional monoclonal antibodies against human TIMP-3 were identified from the pools of hybridomas previously described, using an ELISA with either passively coated TIMP-3 or TIMP-3 anchored via anti-TIMP-3 MAB9731 (R&D Systems). Antibodies that were positive in the ELISA were also tested for TIMP-3 accumulation, using an assay substantially as described previously. Twenty-six hybridomas were identified as secreting antibodies that facilitated accumulation of TIMP-3 when spent hybridoma supernatant fluid was evaluated in a TIMP-3 accumulation assay (after buffer exchange into serum-free medium to reduce background effects on the TIMP-3 accumulation assay). Four of these hybridomas were lost during subcloning, but the remaining 22 were cultured at a larger scale, and monoclonal antibodies were purified and evaluated, for TIMP-3 accumulation and possible effects on TIMP-3 inhibition of MMP-13 (as well as any direct effect on MMP-13 itself).

None of the 22 antibodies evaluated adversely affected the ability of TIMP-3 to inhibit MMP-13, nor did they have an effect upon MMP-13 itself. In the TIMP-3 accumulation assay, the antibodies were evaluated for reproducible, titratable increase in the accumulation of TIMP-3 in supernatant fluid at levels of at least about 1.5 times higher than that observed with a negative control antibody. Four antibodies were selected for further analysis and cell line development; results of a representative TIMP-3 accumulation assay and MMP-13 inhibition assay on these antibodies are shown below. The MMP-13 inhibition assay utilized 1.5 nM MMP-13 (CalBiochem), purified TIMP-3 at 0.25 nM (which resulted in a 54% inhibition of MMP-13 activity in the absence of any antibody), and 20 microM Mca-KPLGL-Dpa-AR-NH2 Fluorogenic Peptide Substrate (ES010 substrate, R & D Systems) in a 100 microL assay volume. For the MMP-13 inhibition assay, antibody 10A7 was included as a control Ab known to compromise TIMP-3 inhibition of MMP-13 by at least a certain degree.

TABLE 9
Effect of anti-TIMP-3 antibodies
on inhibition of MMP-13 by TIMP-3

| | | 16A1.1 | 18H1.1 | 17A4.1 | 18C1.1 | 10A7 |
|---|---|---|---|---|---|---|
| MAb [ ] | | % inhib'n | % inhib'n | % inhib'n | % inhib'n | % inhib'n |
| 20 | nM | 40% | 41% | 45% | 47% | 1% |
| 10 | nM | 47% | 43% | 47% | 45% | 1% |
| 5 | nM | 48% | 37% | 51% | 47% | 4% |
| 2.5 | nM | 50% | 41% | 48% | 47% | 8% |
| 1.25 | nM | 45% | 43% | 43% | 44% | 10% |
| 0.625 | nM | 43% | 40% | 34% | 33% | 11% |
| 0.312 | nM | 45% | 38% | 44% | 47% | 14% |

These results indicate that antibodies 16A1.1, 18H1.1, 17A4.1 and 18C1.1 did not significantly decrease the ability of TIMP-3 to inhibit MMP-13, at concentrations up to about 80-fold molar excess of antibody.

TABLE 10
Effect of anti-TIMP-3 antibodies on Accumulation of TIMP-3

| | | | | | | Fold |
|---|---|---|---|---|---|---|
| | | | | | 2 microM | increase |
| | 2 | 1 | 05 | 025 | negative | over |
| | microM | microM | microM | microM | control | control |
| 16A1.1 | 0.420 | 0.269 | 0.203 | 0.201 | 0.264 | 1.59 |
| 18H1.1 | 0.107 | 0.061 | 0.032 | 0.034 | 0.016 | 6.69 |
| 17A4.1 | 1.516 | 1.146 | 0.68 | 0.744 | 0.57 | 2.66 |
| 18C1.1 | 1.741 | 1.124 | 0.971 | 0.873 | 0.958 | 1.82 |

These results indicate that antibodies 16A1.1, 18H1.1, 17A4.1 and 18C1.1 lead to accumulation of TIMP-3 in the supernatant fluid. Subsequent analysis indicated that antibodies 17A4 and 18C1 had the same amino acid sequence. The remaining 18 clones were stored for possible additional evaluation in the future.

We claim:

1. A TIMP-3 binding protein that binds TIMP-3 and inhibits internalization of TIMP-3 by LRP-1.

2. The TIMP-3 binding protein of claim 1 that is an antibody or an LRP-1 peptide.

3. The TIMP-3 binding protein of claim 1 or claim 2 that decreases the inhibition of MMP-13 by TIMP-3 by less than 30%.

4. A method of increasing TIMP-3 in extracellular matrix by contacting TIMP-3 with a TIMP-3 binding protein according to claim 1 or claim 2.

5. The method of claim 4 wherein the TIMP-3 is contacted with the TIMP-3 binding protein in vivo, ex vivo or in vitro.

6. The method of claim 5 wherein the TIMP-3 is contacted with the TIMP-3 binding protein in vivo by administering the TIMP-3 binding protein to a mammal.

7. A method of treating a mammal afflicted with a condition in which matrix metalloproteinases play a deleterious role, comprising administering a TIMP-3 binding protein according to claim 1 or claim 2 to the mammal.

8. The method of claim 7 wherein the condition is selected from the group consisting of inflammation, cancer, and a condition characterized by excessive degradation of the extracellular matrix.

9. The method of claim 8 wherein the condition is selected from the group consisting of osteoarthritis and congestive heart failure.


SEQUENCE LISTING (2 sequences)

SEQ ID NO: 1 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 2 - protein, 8 aa, Artificial
  FLAG peptide
DYKDDDDK